(19) United States
(12) Patent Application Publication
(10) Pub. No.: US 2025/0273307 A1
(43) Pub. Date: Aug. 28, 2025

(54) TEST ASSIST APPARATUS, TEST ASSIST METHOD, AND RECORDING MEDIUM

(71) Applicant: NEC Corporation, Tokyo (JP)

(72) Inventors: Hirokazu Sakamoto, Tokyo (JP); Tomoharu Kiyuna, Tokyo (JP); Tatsu Kimura, Tokyo (JP)

(73) Assignee: NEC Corporation, Tokyo (JP)

(21) Appl. No.: 19/057,023

(22) Filed: Feb. 19, 2025

(30) Foreign Application Priority Data
Feb. 27, 2024 (JP) 2024-027605

(51) Int. Cl. G16H 10/20; G06N 5/04; G16H 10/40

(52) U.S. Cl. CPC G16H 10/20; G06N 5/04; G16H 10/40

(57) ABSTRACT

This test assist apparatus includes: an acquiring section for acquiring utterance information which indicates the content of an utterance of a patient during a test performed on the patient, state information regarding the feelings of the patient during the test, and basic information regarding the test; an interruption predicting section for predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and an outputting section for outputting the probability of interruption.

### CROSS REFERENCE TO RELATED APPLICATIONS

This application is based upon and claims the benefit of priority from Japanese Patent Application No. 2024-027605 filed on Feb. 27, 2024, the disclosure of which is incorporated herein in its entirety by reference.

### TECHNICAL FIELD

The present disclosure relates to a test assist apparatus, a test assist method, and a recording medium.

### BACKGROUND ART

Medical treatment assist techniques are known. Examples of medical treatment assist techniques include the technique disclosed in Patent Literature 1. Patent Literature 1 discloses a system for assisting medical treatments appropriate for various aspects of a patient. The system disclosed in Patent Literature 1 includes an acquiring section, an analyzing section, and a display control section. The acquiring section acquires patient information regarding a patient, medical interview information indicating the details of the patient's answers to the questions asked in a medical interview, and conversation information having recorded therein a conversation with the patient. The analyzing section performs analytical processing with use of the patient information, the medical interview information, and the conversation information, to generate patient characteristic information indicating the psychological aspect and societal aspect of the patient. The display control section displays, based on the patient characteristic information, the psychological aspect and the societal aspect of the patient such that the psychological aspect and the societal aspect are discriminable from each other.

### CITATION LIST

### Patent Literature

### Patent Literature 1

Japanese Patent Application Publication Tokukai No. 2023-071244

### SUMMARY OF INVENTION

### Technical Problem

In some cases, a patient feels tense and uneasy in a test such as endoscopy. In such cases, the test can be interrupted because, for example, the patient feels a pain. With the technique disclosed in Patent Literature 1, there is a problem of being unable to predict an interruption of a test resulting from the feelings of a patient being tested, such as uneasiness.

The present disclosure has been made in view of the above problem, and an example object thereof is to provide a technique which makes it possible to predict an interruption of a test resulting from the feelings of a patient being tested, such as uneasiness.

### Solution to Problem

A test assist apparatus in accordance with an example aspect of the present disclosure includes at least one processor, and the at least one processor carries out: an acquiring process of acquiring utterance information which indicates content of an utterance of a patient during a test performed on the patient, state information regarding feelings of the patient during the test, and basic information regarding the test; an interruption predicting process of predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and an outputting process of outputting the probability of interruption.

A test assist method in accordance with an example aspect of the present disclosure includes: at least one processor acquiring utterance information which indicates content of an utterance of a patient during a test performed on the patient, state information regarding feelings of the patient during the test, and basic information regarding the test; the at least one processor predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and the at least one processor outputting the probability of interruption.

A recording medium in accordance with an example aspect of the present disclosure is a recording medium having recorded thereon a test assist program for causing a computer to function as a test assist apparatus, and the test assist program causes the computer to carry out: an acquiring process of acquiring utterance information which indicates content of an utterance of a patient during a test performed on the patient, state information regarding feelings of the patient during the test, and basic information regarding the test; an interruption predicting process of predicting a probability of interruption of the test performed on the patient, from the utterance the state information, and the basic information, with use of a prediction model for predicting a probability of interruption of a test, the prediction model being generated by machine learning in which samples of past test interruptions are used as training data; and an outputting process of outputting the probability of interruption.

### Advantageous Effects of Invention

An example aspect of the present disclosure provides an example advantage of making it possible to provide a technique for predicting an interruption of a test resulting from the feelings of a patient being tested, such as uneasiness.

### BRIEF DESCRIPTION OF DRAWINGS

FIG. 1 is a block diagram illustrating a configuration of a test assist apparatus in accordance with the present disclosure.

FIG. 2 is a flowchart illustrating a flow of a test assist method in accordance with the present disclosure.

FIG. 3 is a block diagram illustrating a configuration of an information processing apparatus in accordance with the present disclosure.

FIG. 4 is a diagram illustrating a functional configuration of a control section in accordance with the present disclosure.

FIG. 5 is a diagram illustrating a specific example of output information outputted by an output control section in accordance with the present disclosure.

FIG. 6 is a flowchart illustrating an example flow of a test assist method in accordance with the present disclosure.

FIG. 7 is a block diagram illustrating a configuration of a computer which functions as the test assist apparatus or the information processing apparatus in accordance with the present disclosure.

### EXAMPLE EMBODIMENTS

The following description will discuss example embodiments of the present invention. However, the present invention is not limited to the example embodiments described below, but can be altered by a skilled person in the art within the scope of the claims. For example, any embodiment derived by appropriately combining techniques (some or all of products or methods) adopted in differing example embodiments described below can be within the scope of the present invention. Further, any embodiment derived by appropriately omitting one or more of the techniques adopted in differing example embodiments described below can be within the scope of the present invention. Furthermore, the advantage mentioned in each of the example embodiments described below is an example advantage expected in that example embodiment, and does not define the extension of the present invention. That is, any embodiment which does not provide the example advantages mentioned in the example embodiments described below can also be within the scope of the present invention.

### First Example Embodiment

The following description will discuss a first example embodiment, which is an example embodiment of the present invention, in detail with reference to the drawings. The present example embodiment is basic to each of the example embodiments which will be described later. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### Configuration of Test Assist Apparatus

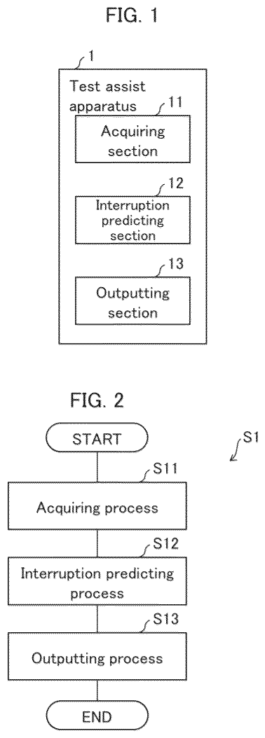

The configuration of a test assist apparatus 1 is described here with reference to FIG. 1. FIG. 1 is a block diagram illustrating the configuration of the test assist apparatus 1. The test assist apparatus 1 includes an acquiring section 11, an interruption predicting section 12, and an outputting section 13, as illustrated in FIG. 1.

The acquiring section 11 acquires utterance information indicating the content of an utterance of a patient during a test performed on the patient, state information regarding the feelings of the patient during the test, and basic information regarding the test. The interruption predicting section 12 predicts a probability of interruption of the test performed on the patient, from the utterance information, the state information, and the basic information, with use of a prediction predicting a probability of interruption of a test, the prediction model being generated by machine learning in which samples of past test interruptions are used as training data. The outputting section 13 outputs the probability of interruption.

### Example Advantage of Test Assist Apparatus

As above, the test assist apparatus 1 includes an acquiring section 11 for acquiring utterance information indicating the content of an utterance of a patient during a test performed on the patient, state information regarding the feelings of the patient during the test, and basic information regarding the test; an interruption predicting section 12 for predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and an outputting section 13 for outputting the probability of interruption. Thus, the test assist apparatus 1 provides an example advantage of making it possible to predict an interruption of a test resulting from the feelings of a patient being tested, such as uneasiness.

### Flow of Test Assist Method

The flow of a test assist method S1 is described here with reference to FIG. 2. FIG. 2 is a flowchart illustrating the flow of the test assist method S1. The test assist method S1 includes an acquiring process S11, an interruption predicting process S12, and an outputting process S13, as illustrated in FIG. 2.

In the acquiring process S11, at least one processor acquires utterance information which indicates the content of an utterance of a patient during a test performed on the patient, state information regarding the feelings of the patient during the test, and basic information regarding the test. In the interruption predicting process S12, the at least one processor predicts a probability of interruption of the test performed on the patient, from the utterance information, the state information, and the basic information, with use of a prediction model for predicting a probability of interruption of a test, the prediction model being generated by machine learning in which samples of past test interruptions are used as training data. In the outputting process S13, the at least one processor outputs the probability of interruption.

### Example Advantage of Test Assist Method

As above, the test assist method S1 includes: an acquiring process S11 of at least one processor acquiring utterance information which indicates the content of an utterance of a patient during a test performed on the patient, state information regarding the feelings of the patient during the test, and basic information regarding the test; an interruption predicting process S12 of the at least one processor predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and an outputting process S13 of the at least one processor outputting the probability of interruption. Thus, the test assist method S1 provides an example advantage of making it possible to predict an interruption of a test resulting from the feelings of a patient being tested, such as uneasiness.

### Second Example Embodiment

The following description will discuss a second example embodiment, which is an example embodiment of the present invention, in detail with reference to the drawings. A component having the same function as a component described in the above example embodiment is assigned the same reference sign, and the description thereof is omitted where appropriate. It should be noted that the applicability of the techniques adopted in the present example embodiment is not limited to the present example embodiment. That is, the techniques adopted in the present example embodiment can be adopted in another example embodiment included in the present disclosure, to the extent of constituting no specific technical obstacle. Further, the techniques illustrated in the drawings referred to for the description of the present example embodiment can be adopted in another example embodiment included in the present disclosure, to the extent of constituting no specific technical obstacle.

### Configuration of Information Processing Apparatus

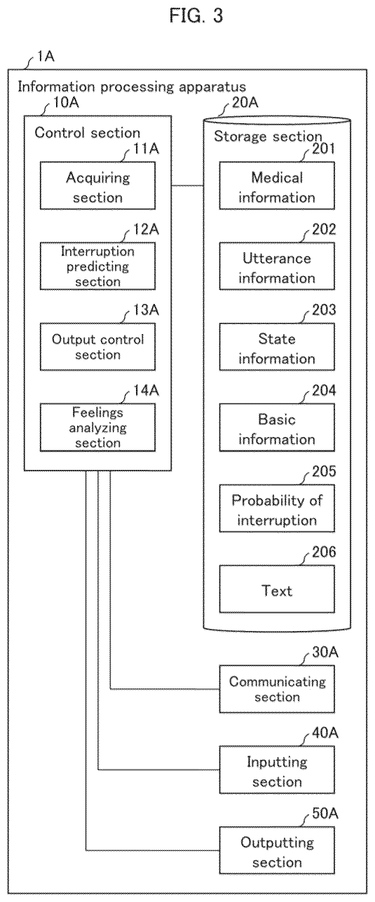

The configuration of an information processing apparatus 1A is described here with reference to FIG. 3. FIG. 3 is a block diagram illustrating the configuration of the information processing apparatus 1A. The information processing apparatus 1A includes a control section 10A, a storage section 20A, a communicating section 30A, an inputting section 40A, and an outputting section 50A.

### Communicating Section

The communicating section 30A communicates with an apparatus external to the information processing apparatus 1A over a communication line. A specific configuration of the communication line does not limit the present example embodiment, but examples of the communication line include a wireless local area network (LAN), a wired LAN, a wide area network (WAN), a public network, a mobile data communication network, and a combination thereof. The communicating section 30A transmits, to another apparatus, data supplied from the control section 10A, and supplies the control section 10A with data received from another apparatus.

### Inputting Section

The inputting section 40A is a component for accepting an input to the information processing apparatus 1A, and includes inputting equipment such as, for example, a keyboard, a mouse, a touch panel, a camera, or a microphone. Further, the inputting section 40A may be a component for accepting data from inputting equipment via an interface such as, for example, a universal serial bus (USB).

### Outputting Section

The outputting section 50A is a component for producing an output from the information processing apparatus 1A, and includes outputting equipment such as, for example, a display, a printer, a touch panel, or a speaker. The outputting section 50A may be a component which, for example, includes an interface such as a USB and outputs data to outputting equipment via the interface.

### Storage Section

In the storage section 20A, various kinds of information to be referred to by the control section 10A are stored. Examples of such information include medical information 201, utterance information 202, state information 203, basic information 204, probability of interruption 205, and text 206.

### Medical Information

The medical information 201 is information regarding medical care for a patient. Examples of the medical information 201 include medical record information. As an example, the medical information 201 includes at least one selected from the group consisting of personal information regarding the patient, information on findings shown by a medical examination performed on the patient, and medical history information regarding the patient. The personal information regarding the patient is personal information regarding the patient, and includes, for example, information indicating the age, the gender, the amount of smoking, etc. of the patient. The information on findings shown by a medical examination performed on the patient is information indicating findings made by a medical service worker, such as a doctor, and includes, for example, information obtained by the medical service worker through a medical interview or an inspection. The information obtained through a medical interview or an inspection may be, for example, text “pressure on the abdomen causes a pain”. The medical history information regarding the patient is information regarding the history of disease of the patient, and includes, for example, information indicating the past medical history of the patient, the past medical history of the family, the amount of smoking of the patient, etc.

The medical information 201 may include a diagnosis target image of a patient. As an example, the diagnosis target image includes at least one of images which are an X-ray image, an endoscope image, a pathological image, an MRI image, and a CT image. In the storage section 20A, respective pieces of medical information 201 regarding a plurality of patients are stored.

### Utterance Information

The utterance information 202 is information indicating the content of an utterance of a patient during a test. For example, the utterance information 202 may be speech data obtained by picking up an utterance of a patient being tested. In this case, examples of the utterance of a patient includes, for example, a complaint and a question made by the patient. The utterance information 202 may be data in text format, or may be speech data. For example, the utterance information 202 is text representing a patient's utterance such as “Is it safe? I am scared. I feel a pain.”

### State Information

The state information 203 is information regarding the feelings of a patient. As an example, the state information 203 includes information which indicates at least one selected from the group consisting of the facial expression, the manner of speaking, a vital sign, and a feelings analysis result of the patient in a test. As an example, the feelings analysis is carried out by a feelings analyzing section 14A, which will be described later. In other words, the state information 203 may include information indicating the result of an analysis, carried out by the feelings analyzing section 14A, of the feelings of the patient. As an example, the information indicating the result of an analysis of the feelings indicates a state of “tenseness” or “uneasiness”.

### Basic Information

The basic information 204 is information regarding a test. As an example, the basic information 204 includes information indicating the type of test (lower endoscopy, etc.) and the phase of test (insertion of an endoscope). Further, the basic information 204 may include information (e.g., “first-ever”, “second”, “third”, etc.) indicating whether a patient has an experience of undergoing the test.

### Probability of Interruption

The probability of interruption 205 is a probability (e.g., 40%) that a test will be interrupted. The probability of interruption 205 is a probability predicted by an interruption predicting section 12A, which will be described later.

### Text

The text 206 is text representing at least one selected from the group consisting of the basis for an interruption of a test performed on a patient and advice on dealing with the interruption. Examples of the text 206 include text “The patient has a strong feeling of uneasiness about undergoing the test for the first time. In some of past similar cases, the uneasy and tense feelings of a patient stiffened their abdomen, and the insertion of an endoscope therefore caused a pain. Additional explanation is highly likely to allay patient's uneasiness.”

### Control Section

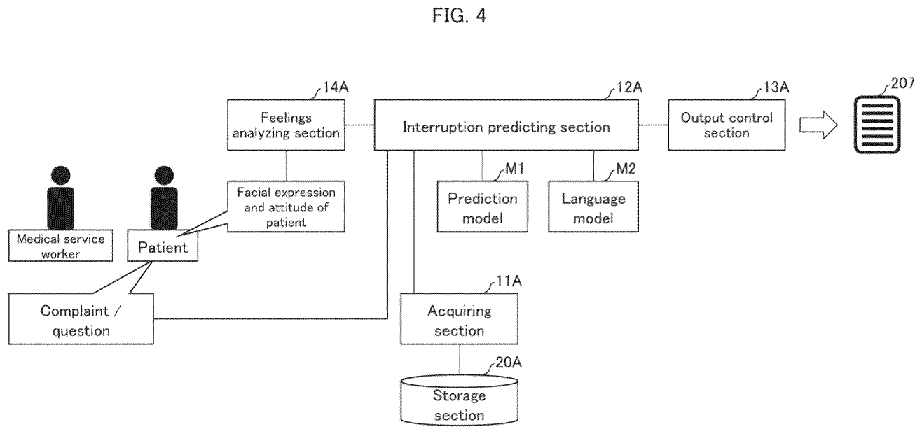

FIG. 4 is a diagram illustrating a functional configuration of the control section 10A. The control section 10A includes an acquiring section 11A, an interruption predicting section 12A, an output control section 13A, and a feelings analyzing section 14A.

### Acquiring Section

The acquiring section 11A acquires the utterance information 202 indicating the content of an utterance of a patient during a test performed on the patient and the basic information 204 regarding the test, and provides the interruption predicting section 12A with the utterance information 202 and the basic information 204 acquired. As an example, the acquiring section 11A may acquire the utterance information 202 and the basic information 204 by retrieving the utterance information 202 and the basic information 204 from a storage location (which may be storage in the information processing apparatus 1A, or may be storage external to the information processing apparatus 1A) designated by a user of the information processing apparatus 1A. The acquiring section 11A may acquire the utterance information 202 and the basic c information 204 by receiving the utterance information 202 and the basic information 204 from another apparatus via the acquire the utterance information 202 and the basic information 204 inputted to the inputting section 40A.

As an example, the acquiring section 11A acquires speech data representing an utterance of a patient picked up during a test, and converts the speech data into the utterance information 202 in text format. In this case, the acquiring section 11A may carry out speaker recognition based on the speech data and transcribe utterances speaker by speaker, to generate the utterance information 202 which indicates the content of an utterance of the patient.

Further, the acquiring section 11A may acquire the medical information 201 in addition to the utterance information 202 and the basic information 204. As an example, the acquiring section 11A may acquire the medical information 201 by retrieving the medical information 201 from a storage location (which may be storage in the information processing apparatus 1A, or may be storage external to the information processing apparatus 1A) designated by a user of the information processing apparatus 1A. The acquiring section 11A may acquire the medical information 201 by receiving the medical information 201 from another apparatus via the acquire the medical information 201 inputted to the inputting section 40A.

### Feelings Analyzing Section

The feelings analyzing section 14A analyzes the feelings of a patient in a test, and generates the result of the analysis. As an example, the feelings analyzing section 14A may analyze at least one selected from the group consisting of speech data regarding the patient picked up in a test and image data captured in the test, to analyze the feelings of the patient. The image data may be data representing a still image, or may be data representing a moving image. Examples of an approach for the feelings analysis may include an approach of using dictionaries prepared in advance and an approach of using a trained model generated by machine learning. In a case of using the trained model, the input to the trained model includes, for example, at least one selected from the group consisting of text, speech data, and image data. The output from the trained model includes information indicating the result of classification of the feelings.

The feelings analyzing section 14A provides the interruption predicting section 12A with the analysis result. The feelings analyzing section 14A may output the analysis result by writing the analysis result in a storage location (which may be storage in the information processing apparatus 1A, or may be storage external to the information processing apparatus 1A) designated by a user of the information processing apparatus 1A. Further, the feelings analyzing section 14A may transmit the analysis result to another apparatus via the communicating section 30A, or may output the analysis result to outputting equipment such as a display.

### Interruption Predicting Section

The interruption predicting section 12A acquires the state information 203. As an example, the interruption predicting section 12A may acquire the state information 203 by retrieving the state information 203 from a storage location (which may be storage in the information processing apparatus 1A, or may be storage external to the information processing apparatus 1A) designated by a user of the information processing apparatus 1A. The interruption predicting section 12A may acquire the state information 203 by receiving the state information 203 from another apparatus via the communicating section 30A. The interruption predicting section 12A may acquire the state information 203 inputted to the inputting section 40A.

As an example, the state information 203 includes the analysis result provided by the feelings analyzing section 14A. Further, the state information 203 may include information inputted by a medical service worker who performs a test on a patient, with use of the inputting section 40A, or information transmitted to the information processing apparatus 1A from a communication apparatus operated by the medical service worker. In this case, the medical service worker performing a test on a patient observes the facial expression, etc. of the patient in the test, and in a case where, for example, it is considered that the patient is feeling tense or uneasy and/or the patient does not understand the explanation, the medical service worker inputs information to that effect.

The state information 203 may include information inputted by the patient with use of the inputting section 40A, or information transmitted to the information processing apparatus 1A from a communication apparatus operated by the patient. In this case, if the patient cannot understand the explanation made by the medical service worker and/or the patient feels tense or uneasy about therapy, the patient inputs information to that effect.

The interruption predicting section 12A predicts the probability of interruption 205 of a test performed on a patient, from the utterance information 202, the state information 203, and the basic information 204, with use of a prediction model M1 for predicting a probability of interruption of a test. The prediction model M1 may be stored in the storage section 20A of the information processing apparatus 1A, or may be stored in an apparatus other than the information processing apparatus 1A. The prediction model M1 being stored in the storage section 20A means that parameters defining the prediction model M1 are stored in the storage section 20A.

The prediction model M1 is a model generated by machine learning in which samples of past test interruptions are used as training data. As an example, the training data includes a plurality of sets each of which is a set of (i) utterance information regarding a patient, the state information, and the basic information regarding a test in the test of the past and (ii) a label which indicates whether the test was interrupted.

As an example, input information inputted by the interruption predicting section 12A to the prediction model M1 includes at least one selected from the group consisting of the utterance information 202 regarding a patient, the state information 203, and the basic information 204 regarding a test in the test. Further, the input information inputted by the interruption predicting section 12A to the prediction model M1 may include the medical information 201. Furthermore, the output from the prediction model M1 includes the probability of interruption 205.

The interruption predicting section 12A generates, from the basic information 204, the utterance information 202, and the state information 203, the text 206 representing at least one selected from the group consisting of the basis for an interruption of a test performed on a patient and advice on dealing with the interruption, with use of a language model M2 generated by machine learning. For example, the language model M2 is a large language model formed by an artificial neural network having a great number of parameters. The language model M2 may be stored in the storage section 20A of the information processing apparatus 1A, or may be stored in an apparatus other than the information processing apparatus 1A. The language model M2 being stored in the storage section 20A means that parameters defining the language model M2 are stored in the storage section 20A.

Examples of the language model M2 includes, but is not limited to, generative AI such as Chat Generative Pre-Trained Transformer (ChatGPT) or Generative Pre-trained Transformer 4 (GPT-4), and such generative AI having been fine-tuned with data related to medical care.

Input information inputted by the interruption predicting section 12A to the language model M2 includes at least one selected from the group consisting of the utterance information 202, the state information 203, and the basic information 204. Further, the input information may include the medical information 201. Furthermore, the input information may include information indicating instructions that the text 206 is to be generated. For example, this information may be text “please tell me the basis for an interruption of the test and advice on dealing with the interruption.” Output information outputted from the language model M2 includes the text 206.

In a case where the language model M2 is stored in an apparatus other than the information processing apparatus 1A, the interruption predicting section 12A inputs the input information to the language model M2 by, for example, transmitting, via the communicating section 30A, the input information to the apparatus in which the language model M2 is stored. In this case, the interruption predicting section 12A receives the information outputted by the language model M2 from that apparatus via the communicating section 30A.

The interruption predicting section 12A may refer to the state information 203 to determine whether the patient is feeling uneasy. In this case, the interruption predicting section 12A may refer to the state information 203 to determine whether the patient is feeling uneasy, and in a case of a determination that the patient is feeling uneasy, the interruption predicting section 12A may predict the probability of interruption and generate the text 206.

### Output Control Section

The output control section 13A outputs output information 207, which includes the probability of interruption 205 predicted by the interruption predicting section 12A and the text 206. As an example, the output control section 13A may output the probability of interruption 205 and the text 206 by writing the probability of interruption 205 and the text 206 in a storage location (which may be storage in the information processing apparatus 1A, or may be storage external to the information processing apparatus 1A) designated by a user of the information processing apparatus 1A. Further, the output control section 13A may transmit the probability of interruption 205 and the text 206 via the communicating section 30A, or may output the probability of interruption 205 and the text 206 to outputting equipment such as a display.

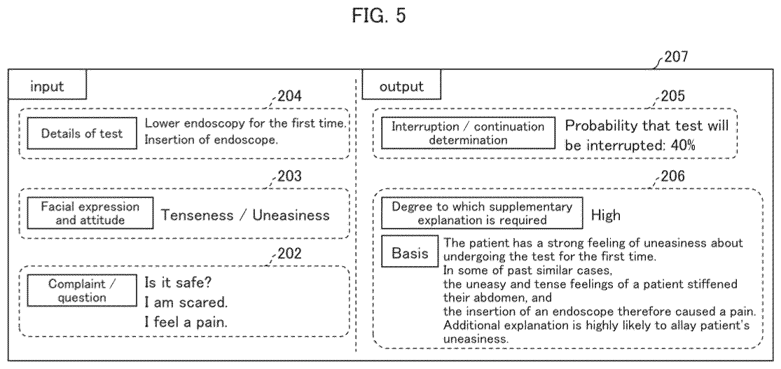

FIG. 5 is a diagram illustrating specific examples of the probability of interruption 205 and the text 206 which are outputted by the output control section 13A. As an example, the output control section 13A may display the output information 207, which includes the probability of interruption 205 and the text 206, on displaying equipment, as illustrated in FIG. 5. In this case, a screen displayed on the displaying equipment includes the probability of interruption 205 and the text 206. The screen further includes the basic information 204, the state information 203, and the utterance information 202, which are inputs to the information processing apparatus 1A. As illustrated in FIG. 5, the output control section 13A may output at least one selected from the group consisting of the basic information 204, the state information 203, and the utterance information 202, together with the probability of interruption 205 and the text 206.

In the example of FIG. 5, the output information 207 includes the basic information 204 in which the “details of test” is “Lower endoscopy for the first time. Insertion of endoscope.” Further, the output information 207 includes the state information 203 in which the “facial expression and attitude” is “tenseness/uneasiness”. Further, the output information 207 includes the utterance information 202 in which the “complaint/question” is “Is it safe? I am scared. I feel a pain.”

In the example of FIG. 5, the output information 207 includes the probability of interruption 205 in which the “interruption/continuation determination” is “Probability that test will be interrupted: 40%”. Further, the output information 207 includes the text 206 in which the “basis” is “The patient has a strong feeling of uneasiness about undergoing the test for the first time. In some of past similar cases, the uneasy and tense feelings of a patient stiffened their abdomen, and the insertion of an endoscope therefore caused a pain. Additional explanation is highly likely to allay patient's uneasiness.”

For example, at least one selected from the group consisting of the probability of interruption 205 and the text 206 is used in decision-making by a medical service worker who performs a test on a patient. As an example, the medical service worker checks the probability of interruption 205 outputted, to decide on an action such as temporarily interrupting the test to make an additional explanation with the text 206 used as a guide, in order to allay the uneasiness of the patient.

### Flow of Test Assist Method

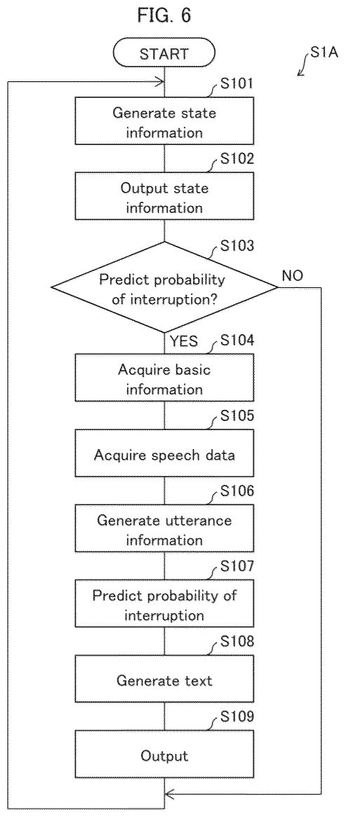

FIG. 6 is a flowchart illustrating an example flow of a test assist method S1A carried out by the information processing apparatus 1A. The steps included in the flowchart of FIG. 6 may be carried out in parallel with each other or in a different order. In step S101, the feelings analyzing section 14A analyzes the facial expression, the content of a speech, and the like of a patient in a test, to generate an analysis result, which is the state information 203.

In step S102, the feelings analyzing section 14A outputs the state information 203, which is the analysis result, to outputting equipment. As an example, the feelings analyzing section 14A may output the state information 203 to a display, and display the state information 203 on the display.

A medical service worker who performs a test on the patient checks the state information 203 displayed on the display, to determine whether to predict the probability of interruption 205. In a case of predicting the probability of interruption 205, the medical service worker operates an operation element or the like which is connected to the inputting section 40A, to input, to the information processing apparatus 1A, an instruction to predict the probability of interruption 205. For example, in a case where the state information 203 outputted includes at least one selected from the group consisting of information indicating “tenseness” and information indicating “uneasiness”, the medical service worker determines that the probability of interruption 205 should be predicted. In a case of a determination that the probability of interruption 205 does not need to be predicted, the medical service worker does not input the prediction instruction. For example, in a case where the state information 203 outputted does not include information indicating “tenseness” or information indicating “uneasiness”, the medical service worker determines that the probability of interruption 205 should not be predicted.

In step S103, the interruption predicting section 12A determines whether to predict the probability of interruption 205. As an example, the interruption predicting section 12A makes this determination according to whether the prediction instruction is inputted by a user such as the medical service worker or the like. In a case of a determination that the probability of interruption 205 should be predicted, the interruption predicting section 12A moves to the process of step S104. In a case of a determination that the probability of interruption 205 should not be predicted, the interruption predicting section 12A skips the processes of step S104 to step S109 and returns to the process of step S101.

A determination approach carried out by the interruption predicting section 12A in step S103 is not limited to the above-described approach, but the interruption predicting section 12A may use another approach to determine whether to predict the probability of interruption 205. As an example, the interruption predicting section 12A may refer to the state information 203 generated by the feelings analyzing section 14A, to determine whether to predict the probability of interruption 205. In this case, as an example, in a case where the state information 203 includes at least one selected from the group consisting of information indicating “tenseness” and information indicating “uneasiness”, the interruption predicting section 12A may determine that the probability of interruption 205 should be predicted. Further, as an example, in a case where the state information 203 does not include information indicating “tenseness” or information indicating “uneasiness”, the interruption predicting section 12A may determine that the probability of interruption 205 should not be predicted.

In step S104, the acquiring section 11A acquires the basic information 204 from the storage section 20A. In step S105, the acquiring section 11A acquires speech data picked up in a test. The speech data is data representing the content of an utterance of a patient during a test. In step S106, the acquiring section 11A performs speaker recognition on the speech data and transcribes utterances speaker by speaker, to generate the utterance information 202 which indicates the content of an utterance of the patient. The acquiring section 11A provides the interruption predicting section 12A with the basic information 204 and the utterance information 202 generated.

In step S107, the interruption predicting section 12A predicts the probability of interruption 205, by inputting the basic information 204, the utterance information 202, and the state information 203 to the prediction model M1. Further, in step S108, the interruption predicting section 12A generates the text 206, by inputting the basic information 204, the utterance information 202, and the state information 203 to the language model M2. In step S109, the output control section 13A outputs the probability of interruption 205 and the text 206. Upon the completion of the process of step S109, the control section 10A returns to the process of step S101.

### Example Advantage of Information Processing Apparatus

As above, the information processing apparatus 1A includes an interruption predicting section 12A for generating, from the basic information 204, the utterance information 202, and the state information 203, text 206 which indicates at least one selected from the group consisting of the basis for an interruption of a test performed on a patient and advice on dealing with the interruption, with use of a language model M2 generated by machine learning, and an output control section 13A outputs the text 206 together with the probability of interruption 205. By, for example, a medical service worker referring to the text 206 outputted and interrupting a test to provide a patient with an additional explanation, it is possible to alleviate uneasiness or tenseness of the patient being tested. This makes it possible to prevent occurrence of a case where, for example, in endoscopy, the uneasy and tense feelings of a patient stiffen their abdomen, and the insertion of an endoscope therefore causes a pain. As above, the information processing apparatus 1A provides an example advantage of making it possible to more smoothly proceed with a test than in a case of proceeding with the test without making an additional explanation.

The information processing apparatus 1A includes a feelings analyzing section 14A for analyzing the feelings of a patient during a test, and the interruption predicting section 12A acquires the state information 203 which includes the analysis result provided by the feelings analyzing section 14A. Thus, with the information processing apparatus 1A, it is possible to make an interruption prediction which incorporates the result of analyzing the feelings of a patient being tested, and by a medical service worker or the like checking the probability of interruption 205 outputted, it is possible to make a response such as temporarily interrupting the test to provide the patient with an additional explanation. This provides an example advantage of, for example, making it possible to more smoothly proceed with a test.

In the information processing apparatus 1A, the acquiring section 11A acquires speech data which represents a speech picked up during a test performed on a patient, and converts the speech data into the utterance information 202 in text format. Thus, the information processing apparatus 1A provides an example advantage of making it possible to make an interruption prediction which incorporates the content of an utterance of a patient being tested.

In the information processing apparatus 1A, the interruption predicting section 12A refers to the state information 203 to determine whether a patient is feeling uneasy, and predicts the probability of interruption 205 in a case of a determination that the patient is feeling uneasy. Thus, with the information processing apparatus 1A, it is possible to prevent interruption prediction to be made unnecessarily.

In the information processing apparatus 1A, the state information 203 includes at least one selected from the group consisting of the facial expression, the manner of speaking, a vital sign, and a feelings analysis result of a patient in a test. Thus, with the information processing apparatus 1A, it is possible to make an interruption prediction which incorporates information indicating at least one selected from the group consisting of the facial expression, the manner of speaking, a vital sign, and a feelings analysis result of a patient being tested.

In the information processing apparatus 1A, the probability of interruption 205 is used in decision-making by a medical service worker who performs a test on a patient. Thus, the information processing apparatus 1A provides an example advantage of making it possible for a medical service worker who performs a test to more properly carry out decision-making.

### Software Implementation Example

Some or all of the functions of the test assist apparatus 1 and the information processing apparatus 1A (hereinafter, also referred to as “each apparatus above”) may be implemented by hardware such as an integrated circuit (IC chip), or may be implemented by software.

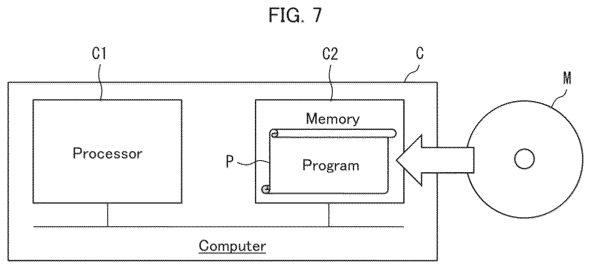

In the latter case, the each apparatus above is provided by, for example, a computer that executes instructions of a program that is software implementing the functions. An example (hereinafter, computer C) of such a computer is illustrated in FIG. 7. FIG. 7 is a block diagram illustrating a hardware configuration of the computer C which functions as each apparatus above.

The computer C includes at least one processor C1 and at least one memory C2. The memory C2 has recorded thereon a program P for causing the computer C to operate as each apparatus above. The processor C1 of the computer C retrieves the program P from the memory C2 and executes the program P, so that the functions of each apparatus above are implemented.

Examples of the processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which inputting-outputting equipment such as a keyboard, a mouse, a display, or a printer is connected.

The program P can be recorded on a non-transitory tangible recording medium M capable of being read by the computer C. Examples of such a recording medium M can tape, a disk, a card, a semiconductor memory, and a programmable logic circuit. The computer C can obtain the program P via such a recording medium M. The program P can be transmitted via a transmission medium. Examples of such a transmission medium can include a communication network and a broadcast wave. The computer C can obtain the program P also via such a transmission medium.

The above-described functions of each apparatus above may be implemented by a single processor provided in a single computer, may be implemented by the cooperation among a plurality of processors provided in a single computer, or may be implemented by the cooperation among a plurality of processors provided in a plurality of respective computers. Further, the program for causing each apparatus above to implement the above-described functions may be stored in a single memory provided in a single computer, may be stored in a distributed manner in a plurality of memories provided in a single computer, or may be stored in a distributed manner in a plurality of memories provided in a plurality of respective computers.

### Additional Remark 1

The whole or part of the example embodiments disclosed above can be described as, but not limited to, the following supplementary notes, and the present invention can be altered in various ways by a skilled person in the art within the scope of the claims.

### Additional Remark A

The whole or part of the example embodiments disclosed above can be described as, but not limited to, the following supplementary notes, and the present invention can be altered in various ways by a skilled person in the art within the scope of the claims.

### Supplementary Note A1

A test assist apparatus, including:
an acquiring means for acquiring utterance information which indicates content of an utterance of a patient during a test performed on the patient, state information regarding feelings of the patient during the test, and basic information regarding the test;
an interruption predicting means for predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting means for outputting the probability of interruption.

### Supplementary Note A2

The test assist apparatus described in supplementary note A1, further including
a text generating means for generating, from the basic information, the utterance information, and the state information, text which represents at least one selected from the group consisting of basis for an interruption of a test performed on the patient and advice on dealing with the interruption, with use of a language model generated by machine learning,
the outputting means being configured to output the text in addition to the probability of interruption.

### Supplementary Note A3

The test assist apparatus described in supplementary note A1 or A2, further including
a feelings analyzing means for analyzing feelings of the patient in the test,
the acquiring means being configured to acquire the state information which includes an analysis result provided by the feelings analyzing means.

### Supplementary Note A4

The test assist apparatus described in any one of supplementary notes A1 to A3, in which
the acquiring means is configured to acquire speech data representing a speech picked up during the test performed on the patient, and convert the speech data into the utterance information in text format.

### Supplementary Note A5

The test assist apparatus described in any one of supplementary notes A1 to A4, in which the interruption predicting means is configured to refer to the state information to determine whether the patient is feeling uneasy, and predict the probability of interruption in a case of a determination that the patient is feeling uneasy.

### Supplementary Note A6

The test assist apparatus described in any one of supplementary notes A1 to A5, in which
the state information includes information which indicates at least one selected from the group consisting of a facial expression, a manner of speaking, a vital sign, and a feelings analysis result of the patient in the test.

### Supplementary Note A7

The test assist apparatus described in any one of supplementary notes A1 to A6, in which
the probability of interruption is used in decision-making by a medical service worker who performs a test on the patient.

### Additional Remark B

The whole or part of the example embodiments disclosed above can be described as, but not limited to, the following supplementary notes, and the present invention can be altered in various ways by a skilled person in the art within the scope of the claims.

### Supplementary Note B1

A test assist method, including:
at least one processor acquiring utterance information which indicates content of an utterance of a patient during a test performed on the patient, state information regarding feelings of the patient during the test, and basic information regarding the test;
the at least one processor predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
the at least one processor outputting the probability of interruption.

### Supplementary Note B2

The test assist method described in supplementary note B1, further including
the at least one processor generating, from the basic information, the utterance information, and the state information, text which represents at least one selected from the group consisting of basis for an interruption of a test performed on the patient and advice on dealing with the interruption, with use of a language model generated by machine learning,
in the outputting, the at least one processor outputting the text in addition to the probability of interruption.

### Supplementary Note B3

The test assist method described in supplementary note B1 or B2, further including
the at least one processor analyzing feelings of the patient in the test,
in the acquiring, the at least one processor acquiring the state information which includes an analysis result provided by the analyzing.

### Supplementary Note B4

The test assist method described in any one of supplementary notes B1 to B3, in which
in the acquiring, the at least one processor acquires speech data representing a speech picked up during the test performed on the patient, and converts the speech data into the utterance information in text format.

### Supplementary Note B5

The test assist method described in any one of supplementary notes B1 to B4, in which
in the predicting, the at least one processor refers to the state information to determine whether the patient is feeling uneasy, and predicts the probability of interruption in a case of a determination that the patient is feeling uneasy.

### Supplementary Note B6

The test assist method described in any one of supplementary notes B1 to B5, in which
the state information includes information which indicates at least one selected from the group consisting of a facial expression, a manner of speaking, a vital sign, and a feelings analysis result of the patient in the test.

### Supplementary Note B7

The test assist method described in any one of supplementary notes B1 to B6, in which
the probability of interruption is used in decision-making by a medical service worker who performs a test on the patient.

### Additional Remark C

The whole or part of the example embodiments disclosed above can be described as, but not limited to, the following supplementary notes, and the present invention can be altered in various ways by a skilled person in the art within the scope of the claims.

### Supplementary Note C1

A test assist program for causing a computer to function as a test assist apparatus,
the test assist program causing the computer to function as:
an acquiring means for acquiring utterance information which indicates content of an utterance of a patient during a test performed on the patient, state information regarding feelings of the patient during the test, and basic information regarding the test;
an interruption predicting means for predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting means for outputting the probability of interruption.

### Supplementary Note C2

The test assist program described in supplementary note C1, further causing the computer to function as
a text generating means for generating, from the basic information, the utterance information, and the state information, text which represents at least one selected from the group consisting of basis for an interruption of a test performed on the patient and advice on dealing with the interruption, with use of a language model generated by machine learning,
the outputting means being configured to output the text in addition to the probability of interruption.

### Supplementary Note C3

The test assist program described in supplementary note C1 or C2, further causing the computer function as
a feelings analyzing means for analyzing feelings of the patient in the test,
the acquiring means being configured to acquire the state information which includes an analysis result provided by the feelings analyzing means.

### Supplementary Note C4

The test assist program described in any one of supplementary notes C1 to C3, in which
the acquiring means is configured to acquire speech data representing a speech picked up during the test performed on the patient, and convert the speech data into the utterance information in text format.

### Supplementary Note C5

The test assist program described in any one of supplementary notes C1 to C4, in which
the interruption predicting means is configured to refer to the state information to determine whether the patient is feeling uneasy, and predict the probability of interruption in a case of a determination that the patient is feeling uneasy.

### Supplementary Note C6

The test assist program described in any one of supplementary notes C1 to C5, in which
the state information includes information which indicates at least one selected from the group consisting of a facial expression, a manner of speaking, a vital sign, and a feelings analysis result of the patient in the test.

### Supplementary Note C7

The test assist program described in any one of supplementary notes C1 to C6, in which
the probability of interruption is used in decision-making by a medical service worker who performs a test on the patient.

### Additional Remark D

The whole or part of the example embodiments disclosed above can be described as, but not limited to, the following supplementary notes, and the present invention can be altered in various ways by a skilled person in the art within the scope of the claims.

### Supplementary Note D1

A test assist apparatus, including at least one processor, the at least one processor carrying out:
an acquiring process of acquiring utterance information which indicates content of an utterance of a patient during a test performed on the patient, state information regarding feelings of the patient during the test, and basic information regarding the test;
an interruption predicting process of predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting process of outputting the probability of interruption.

The test assist apparatus may further include a memory. The memory may have stored therein a program for causing the at least one processor to carry out each of the processes.

### Supplementary Note D2

The test assist apparatus described in supplementary note D1, in which the at least one processor further carries out
a text generating process of generating, from the basic information, the utterance information, and the state information, text which represents at least one selected from the group consisting of basis for an interruption of a test performed on the patient and advice on dealing with the interruption, with use of a language model generated by machine learning, and
in the outputting process, the at least one processor outputs the text in addition to the probability of interruption.

### Supplementary Note D3

The test assist apparatus described in supplementary note D1 or D2, in which the at least one processor further carries out
a feelings analyzing process of analyzing feelings of the patient in the test, and
in the acquiring process, the at least one processor acquires the state information which includes an analysis result provided by the feelings analyzing process.

### Supplementary Note D4

The test assist apparatus described in any one of supplementary notes D1 to D3, in which
in the acquiring process, the at least one processor acquires speech data representing a speech picked up during the test performed on the patient, and converts the speech data into the utterance information in text format.

### Supplementary Note D5

The test assist apparatus described in any one of supplementary notes D1 to D4, in which
in the interruption predicting process, the at least one processor refers to the state information to determine whether the patient is feeling uneasy, and predicts the probability of interruption in a case of a determination that the patient is feeling uneasy.

### Supplementary Note D6

The test assist apparatus described in any one of supplementary notes D1 to D5, in which
the state information includes information which indicates at least one selected from the group consisting of a facial expression, a manner of speaking, a vital sign, and a feelings analysis result of the patient in the test.

### Supplementary Note D7

The test assist apparatus described in any one of supplementary notes D1 to D6, in which
the probability of interruption is used in decision-making by a medical service worker who performs a test on the patient.

### Additional Remark E

The whole or part 41 the example embodiments disclosed above can be described as, but not limited to, the following supplementary notes, and the present invention can be altered in various ways by a skilled person in the art within the scope of the claims.

### Supplementary Note E1

A non-transitory recording medium having recorded thereon a test assist program for causing a computer to function as a test assist apparatus,
the test assist program causing the computer to carry out:
an acquiring process of acquiring utterance information which indicates content of an utterance of a patient during a test performed on the patient, state information regarding feelings of the patient during the test, and basic information regarding the test;
an interruption predicting process of predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting process of outputting the probability of interruption.

### Reference Signs List

1: Test assist apparatus
1A: Information processing apparatus
11, 11A: Acquiring section
12, 12A: Interruption predicting section
13, 50A: Outputting section
13A: Output control section
14A: Feelings analyzing section

1. A test assist apparatus, comprising
at least one processor, the at least one processor carrying out:
an acquiring process of acquiring utterance information which indicates content of an utterance of a patient during a test performed on the patient, state information regarding feelings of the patient during the test, and basic information regarding the test;
an interruption predicting process of predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting process of outputting the probability of interruption.

2. The test assist apparatus according to claim 1,
the at least one processor further carries out a text generating process of generating, from the basic information, the utterance information, and the state information, text which represents at least one selected from the group consisting of basis for an interruption of a test performed on the patient and advice on dealing with the interruption, with use of a language model generated by machine learning, and
in the outputting process, the at least one processor outputs the text in addition to the probability of interruption.

3. The test assist apparatus according to claim 1, wherein
the at least one processor further carries out a feelings analyzing process of analyzing feelings of the patient in the test, and
in the acquiring process, the at least one processor acquires the state information which includes an analysis result provided by the feelings analyzing process.

4. The test assist apparatus according to claim 1,
in the acquiring process, the at least one processor acquires speech data representing a speech picked up during the test performed on the patient, and converts the speech data into the utterance information in text format.

5. The test assist apparatus according to claim 1, wherein
in the interruption predicting process, the at least one processor refers to the state information to determine whether the patient is feeling uneasy, and predicts the probability of interruption in a case of a determination that the patient is feeling uneasy.

6. The test assist apparatus according to claim 1, wherein
the state information includes information which indicates at least one selected from the group consisting of a facial expression, a manner of speaking, a vital sign, and a feelings analysis result of the patient in the test.

7. The test assist apparatus according to claim 1,
the probability of interruption is used in decision-making by a medical service worker who performs a test on the patient.

8. A test assist method, comprising:
at least one processor acquiring utterance information which indicates content of an utterance of a patient during a test performed on the patient, state information regarding feelings of the patient during the test, and basic information regarding the test;
the at least one processor predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
the at least one processor outputting the probability of interruption.

9. A computer-readable non-transitory recording medium having recorded thereon a test assist program for causing a computer to function as a test assist apparatus, the test assist program causing the computer to carry out:
an acquiring process of acquiring utterance information which indicates content of an utterance of a patient during a test performed on the patient, state information regarding feelings of the patient during the test, and basic information regarding the test;
an interruption predicting process of predicting a probability of interruption of the test performed on the patient, from the utterance information, the state information, and the basic information, with use of a prediction model for predicting a probability of interruption of a test, the prediction model being generated by machine learning in which samples of past test interruptions are used as training data; and
an outputting process of outputting the probability of interruption.
